(19) United States
(12) Patent Application Publication
(10) Pub. No.: US 2019/0309017 A1
(43) Pub. Date: Oct. 10, 2019

(54) GLUTAMINASE INHIBITORS

(71) Applicant: Glutaminax, LLC, Rancho Santa Fe, CA (US)

(72) Inventor: Gerald F. Swiss, Rancho Santa Fe, CA (US)

(73) Assignee: Glutaminax, Inc., Rancho Santa Fe, CA (US)

(21) Appl. No.: 16/153,532

(22) Filed: Oct. 5, 2018

Related U.S. Application Data
(60) Provisional application No. 62/736,329, filed on Sep. 25, 2018.
(60) Provisional application No. 62/655,005, filed on Apr. 9, 2018.

(51) Int. Cl. C07K 5/062; A61P 35/00

(52) U.S. Cl. CPC C07K 5/06026; A61K 38/00; A61P 35/00

(57) ABSTRACT

Disclosed are newly identified chemotherapeutic agents. These inhibitors are particularly useful in the treatment of cancers such as leukemic cells and pancreatic cancers.

### CROSS-REFERENCE TO RELATED APPLICATIONS

This application claims priority to U.S. Provisional Application Nos. 62/655,005, filed Apr. 9, 2018; and 62/736,329, filed Sep. 25, 2018; each of which is incorporated herein by reference in its entirety.

### FIELD OF THE INVENTION

This invention is directed to newly identified glutaminase inhibitors. These inhibitors are particularly useful in the treatment of glutamine dependent cancers such as leukemic cells, triple negative breast cancer and pancreatic cancer. In one embodiment, these glutaminase inhibitors are oligopeptides of from 2 to 6 amino acids in length. Preferably, these oligopeptides do not cross the blood brain barrier. This invention is also directed to pharmaceutical compositions comprising such inhibitors as well as methods for using such inhibitors.

### STATE OF THE ART

Amino acid transporters are necessary for cell survival in many mammals including humans. Amino acids are generally classified as essential or non-essential. Essential amino acids are those that are not synthesized in the cells of these mammals and must be absorbed from the nutrients ingested. Non-essential amino acids fall into two categories. The first refers to certain amino acids that are synthesized in vivo but, under certain circumstances, such cells cannot always make amounts sufficient for their immediate needs. The incremental amount of amino acids required by these cells must be absorbed from nutrients ingested. For the sake of clarity, these amino acids are referred to herein as semi-essential amino acids. Finally, those amino acids that are synthesized in amounts that meet the full requirements of the cells are referred to as fully non-essential amino acids. As to the first two categories, the amounts of these amino acids required at a given time is satisfied by amino acid transporters located on the cells.

Amino acid transporters typically transport mono-, di- and tri-peptides (1, 2 or 3 amino acids in length) across the cellular wall and into the intracellular domain. Once so incorporated, intracellular di- and tri-pepsidases degrade these peptides into individual amino acids. These amino acids are then incorporated into the active site of enzymes specific for each amino acid where they are either incorporated into a peptide chain or converted from one amino acid to another.

The identification of amino acid transporters is rapidly growing and certain amino acid transporters are newly recognized both for normal cellular function as well as in aberrant (cancer) cells. For example, the SLC6A14 amino acid transporter is recognized to absorb both neutral amino acids such as glutamine as well as cationic amino acids such as lysine and arginine in normal cells. However, this transporter is the most highly upregulated transporter in many cancers including acute lymphoblastic leukemia (ALL) and pancreatic cancer.

Such an increase in cellular uptake is due to the fact that cancer cell metabolism differs significantly from normal cells especially in the significant increase in the amount of glutamine required as well as in how this amino acid is utilized intracellularly. For example, in glutamine dependent cancers, glutamine is used as a precursor to the amino acid glutamate via the mitochondrial enzyme glutaminase. The art recognizes that inhibition of this enzyme is useful in treating glutamine dependent cancers. www.calithera.com/glutaminase-inhibitor-cb-839/. However, the art further recognizes that glutaminase inhibitors that cross the blood brain barrier have adverse effects on the central nervous system and, as such, are unacceptable.

Accordingly, there remains a need to provide for glutaminase inhibitors that target cancer cells exhibiting a demand for glutamine that are incapable of crossing the blood brain barrier. Identification of such inhibitors would be of great value to the treatment of such cancers.

### SUMMARY OF THE INVENTION

This invention is directed, in part, to the discovery of target specific glutaminase inhibitors designed to inhibit penetration across the blood brain barrier. These inhibitors block the conversion of glutamine to glutamate thereby preventing the utilization of glutamine by glutamine dependent cancer cells. When so inhibited, the cancer cells die.

In one embodiment, the inhibitors of this invention utilize the demand for glutamine by certain cancers to provide for glutamine mimetics that are absorbed by glutamine amino acid transporters and then are incorporated into glutamine related enzymes where they inhibit these enzymes. The inhibitors of this invention are also designed to inhibit their transport across the blood brain barrier thereby inhibiting untoward adverse events in the CNS.

In one embodiment, the inhibitors of this invention are represented by formula I:

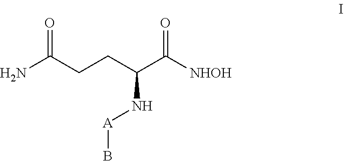

where A is either a covalent bond or from 1 to 6 amino acid residues and B is an amino acid residue provided that at least one of A or B is a glutamic acid or aspartic acid residue; and

pharmaceutically acceptable salts and/or solvates thereof.

In one embodiment, A is either a covalent bond or a single amino acid residue.

In one embodiment, the inhibitors of this invention are represented by formula II(a) and II(b):

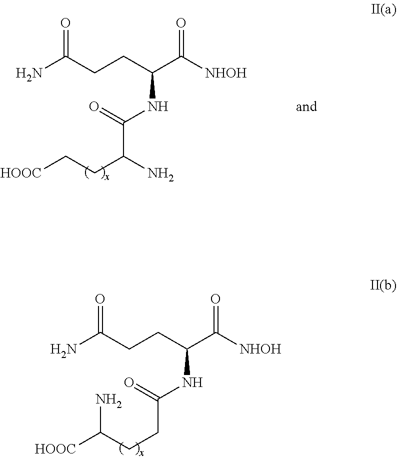

where x is zero, 1, 2, or 3 as well as pharmaceutically acceptable salts and/or solvates thereof. Preferably, x is zero or 1.

As is recognized in the art, the structure of the above compounds II(a) can be abbreviated from the N to the C termini as α-Glu-Gln (NHOH mimetic) when x is 1; and as α-Asp-Gln (NHOH mimetic) when x is zero. The structure of the above compounds II(b) can be abbreviated from the N to the C termini as β-Glu-Gln (NHOH mimetic) when x is 1; and as β-Asp-Gln (NHOH mimetic) when x is zero.

In one embodiment, this invention provides for a pharmaceutical composition comprising an effective amount of a formula I and/or formula II(a)/II(b) as well as a pharmaceutically acceptable excipient.

In one embodiment, this invention provides for a method for inhibiting glutaminase which method comprises contacting said enzyme with an effective amount of a compound of formula I and/or formula II(a)/II(b).

In one embodiment, this invention provide for a method for treating a glutamine dependent cancer in a patient which method comprises:

a) confirming that the cancer is glutamine dependent; and

b) administering to said patient an effective amount of a compound of formula I and/or II(a)/II(b) or a pharmaceutical composition comprising an effective amount of a formula I and/or II(a)/II(b) and a pharmaceutically acceptable excipient.

In one embodiment, the glutamine dependent cancer overexpresses the SLC6A14 amino acid transporter.

### BRIEF DESCRIPTION OF THE DRAWINGS

FIG. 1. The full NMR spectrum for Compound 6.

FIG. 2. The full NMR spectrum for Compound 9.

FIG. 3. The full NMR spectrum for Compound 10.

### DETAILED DESCRIPTION OF THE INVENTION

This invention provides for newly identified glutaminase inhibitors that are useful in the treatment of glutamine dependent cancers.

However, prior to describing this invention in further detail, the following terms will first be defined.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “glutamine dependent cancer” refers to those cancers whose cells require excess amounts of glutamine uptake as compared to normal cells and, as such, overexpress at least one glutamine amino acid transporter on it surface.

The term “amino acid” comprises the 20 naturally occurring L amino acids well known in the art as well as the rare naturally occurring L amino acids and certain synthetic amino acids. The 20 naturally occurring amino acids are identified by three letter or one letter abbreviations as follows:

| Full Name | 3 Letter Abbreviation | 1 Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The rare amino acids include post-translational modifications to the amino acids such as 4-hydroxyproline, deiminated arginine, methylated amino acids such as lysine(Me), lysine(Me2), lysine(Me3), arginine(Me), arginine(Me2) asymmetrical, and arginine(Me2) symmetrical, phosphorylated amino acids such as phosphorylated serine, phosphorylated threonine, and phosphorylated tyrosine as well as others known in the art.

Synthetic amino acids include phenylglycine, 4-hydroxyphenylglycine, 2-amino-6-carboxyhexanoic acid (HOOCCH₂CH₂CH₂CH(NH₂)COOH) and L-amino acids with the following sidechains: 2-hydroxylethyl, 2-thiolethyl; 2-phenylethyl, 2-(4-hydroxyphenyl)ethyl, 3-methylthio-n-propyl, and the like.

“Side chains of amino acids” refer to the R¹⁰ group in the following structure:

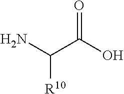

as well as the pyrrolidinyl group of proline.

An “amino acid residue” refers to any one of the following:

a) NH₂CH(R¹⁰)C(O)— if the residue is found at the N-terminus of the peptide;

b) —NHCH(R¹⁰)C(O)— if the residue is found in the peptide chain and neither at the N- or C-termini;

c) —NHCH(R¹⁰)C(O)OH if the residue is found at the C-terminus of the peptide;

d)

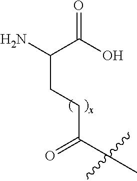

where x is zero or 1 and where the residue is found at the N-terminus of the peptide;

e)

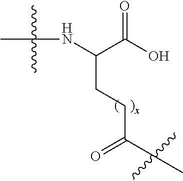

where x is zero or 1 and where the residue is found in the peptide chain and neither the N- or C-termini of the peptide; and

f)

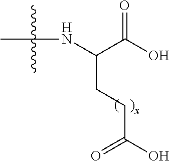

where x is zero or 1 and where the residue is found at the C-terminus of the peptide;

all inclusive of salts and/or solvates thereof.

The term “peptide” as used herein refers to a chain of amino acids that are 2 amino acids in length (sometimes referred to as a “dipeptide”), or 3 amino acids in length (sometimes referred to as a “tripeptide”), or from 2 to 8 amino acids in length (sometimes referred to as an “oligopeptide”).

The compounds of this invention may exist as solvates, especially hydrates. Hydrates may form during manufacture of the compounds or compositions comprising the compounds, or hydrates may form over time due to the hygroscopic nature of the compounds. Compounds of this invention may exist as organic solvates as well, including DMF, ether, and alcohol solvates among others. The identification and preparation of any particular solvate is within the skill of the ordinary artisan of synthetic organic or medicinal chemistry.

“Subject” refers to a mammal. The mammal can be a human or non-human animal mammalian organism.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or causing regression of the disease or disorder.

“Effective amount” refers to the amount of a compound of this invention that is sufficient to treat the disease or disorder afflicting a subject.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

As used herein, the term “pharmaceutically acceptable salts” of compounds disclosed herein are within the scope of the present invention include acid or base addition salts which retain the desired pharmacological activity and is not biologically undesirable (e.g., the salt is not unduly toxic, allergenic, or irritating, and is bioavailable). When the compound of the present invention has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present invention has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

The terms “alpha” and “beta” carboxyl groups on glutamic and aspartic acid are as depicted below where x is 1 (glutamic acid) or zero (aspartic acid):

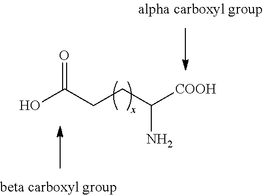

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or d(1) stereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### Synthesis of Representative Compounds of the Invention

In one general embodiment, the synthesis of a compound of formula II(b) where x is one is provided in the reaction scheme below:

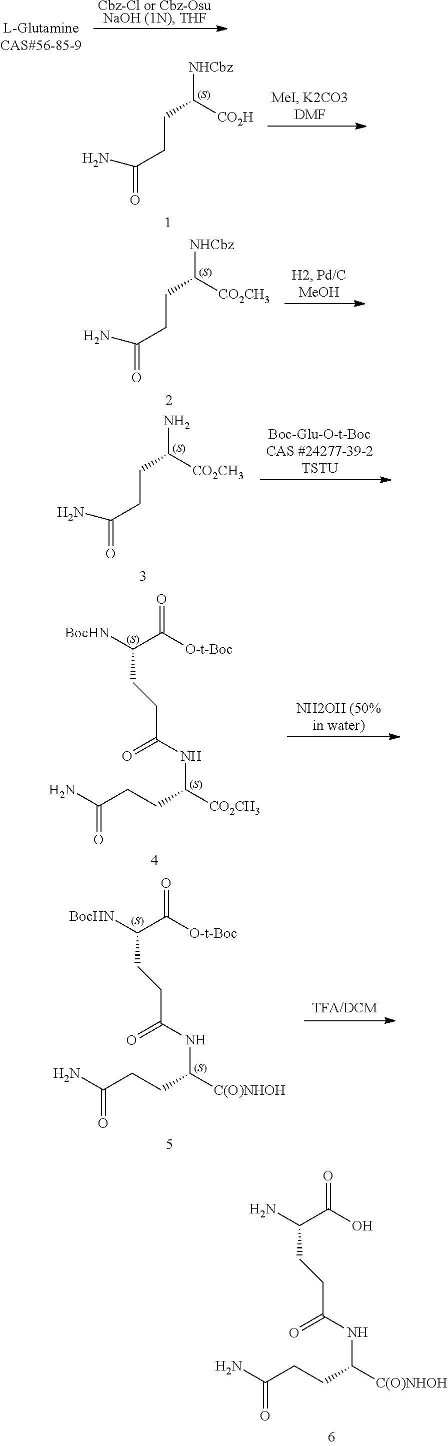

The first three steps of this reaction scheme involve conversion of glutamine into glutamine methyl ester (compound 3). This conversion first entails Cbz protection of the amino group of glutamine by conventional methods to provide for N-Cbz glutamine (compound 1). For example, Cbz-Cl is reacted with glutamine in a suitable inert solvent such as tetrahydrofuran in the presence of a base such as 1 N NaOH to scavenge the acid generated during the reaction. The reaction proceeds for a period of time sufficient until it is substantially complete. The resulting product, N-Cbz glutamine (compound 1), can be recovered by any of a number of conventional steps including, by way of example only, evaporation, chromatography, precipitation, and the like. Alternatively, the product is not recovered but is used directly in the next step.

Next, the carboxyl group of N-Cbz glutamine (compound 1) is converted to the corresponding N-Cbz glutamine methyl ester (compound 2). This conversion follows esterification conditions well known in the art. For example, N-Cbz glutamine (compound 1) is reacted with methyl iodide in a suitable inert solvent such as N,N-dimethylformamide in the presence of a base such as potassium carbonate to scavenge the acid generated during the reaction. The reaction proceeds for a period of time sufficient until it is substantially complete. The resulting product, N-Cbz glutamine methyl ester (compound 2), can be recovered by any of a number of conventional steps including, by way of example only, evaporation, chromatography, precipitation, and the like. Alternatively, the product is not recovered but is used directly in the next step.

In the next step, the Cbz group of compound 2 is removed by conventional hydrogenation conditions to provide for glutamine methyl ester (compound 3) which is recovered and purified by conventional methods such as chromatography, high performance liquid chromatography (HPLC), crystallization, precipitation and the like. Alternatively, glutamine methyl ester (compound 3) is commercially available from SigmaAldrich, St. Louis, Mo., USA.

Glutamine methyl ester (compound 3) is then used in two different reaction schemes to provide for compounds II(a) and II(b) where x is 1. As to the synthesis of compound II(b), commercially available N-Boc Glu-α-t-butyl ester is contacted with glutamine methyl ester (compound 3) under amidation conditions described above to provide for compound 4. Specifically, compound 3 is contacted with N-Boc Glu-alpha-t-butyl ester in an inert solvent such as methylene chloride, toluene, tetrahydrofuran, ethyl acetate and the like in the presence of TSTU (commercially available from SigmaAldrich, St. Louis, Mo., USA as CAS 105832-38-0) under conditions to form compound 4. Subsequently, the methyl ester of the glutamine residue of compound 4 is contacted with hydroxylamine under conditions that convert the methyl carboxylate to the corresponding N-hydroxyamide of compound 5.

In the final steps, the blocking groups are removed by conventional methods to provide for compound 6.

In one general embodiment, the synthesis of a compound of formula II(a) where x is one is provided in the reaction scheme below:

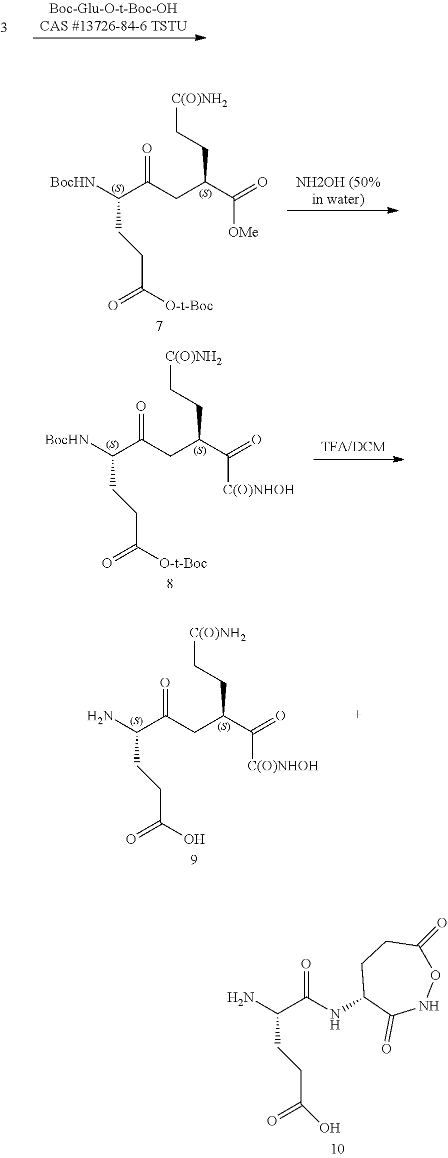

As to the synthesis of compound II(a), commercially available N-Boc Glu-β-t-butyl ester is contacted with glutamine methyl ester (compound 3) under amidation conditions described above to provide for compound 7. Specifically, compound 3 is contacted with N-Boc Glu-β-t-butyl ester in an inert solvent such as methylene chloride, toluene, tetrahydrofuran, ethyl acetate and the like in the presence of TSTU (commercially available from SigmaAldrich, St. Louis, Mo., USA as CAS 105832-38-0) under conditions to form compound 7. Subsequently, the methyl ester of the glutamine residue of compound 7 is contacted with hydroxylamine under conditions that convert the methyl carboxylate to the corresponding N-hydroxyamide of compound 8.

In the final steps, the blocking groups are removed by conventional methods to provide for compound 9.

In this reaction, cyclization of the amide side chain with the hydroxylamide of compound 9 coupled with the loss of ammonia occurs to provide for a side product characterized by compound 10.

Each of compounds 7, 8, 9, and 10 can be isolated and/or purified by conventional methods such as precipitation, crystallization, chromatography, high performance liquid chromatography (HPLC) and other methods well known in the art. The purified compound can be subjected to mass spectroscopy, nuclear magnetic resonance (including COSY and ROSY), C¹⁴ nuclear magnetic resonance, and the like to confirm that the structure of the compound is correct.

Alternatively, the compounds of this invention can be prepared as shown in the scheme below:

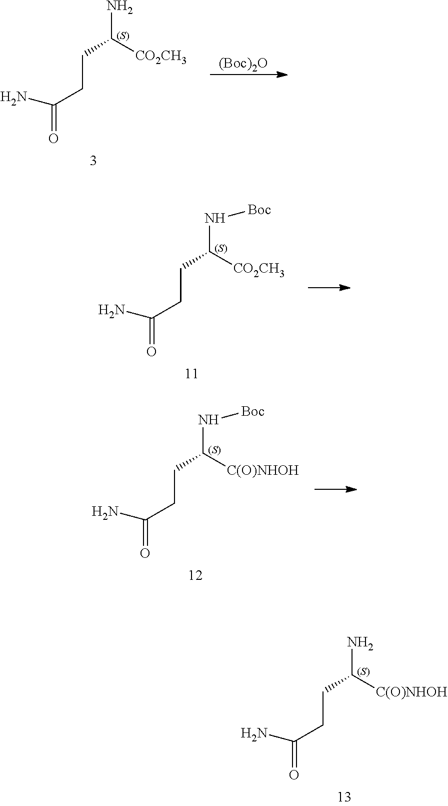

In the above scheme, glutamine methyl ester, compound 3, is first N-protected with a Boc protecting group. The reaction incorporating this protecting group onto the amine is well known in the art. In one embodiment, the reaction employs Boc anhydride (Boc)₂O and the reaction can be conducted in dioxane/water in the presence of sodium dicarbonate. The reaction proceeds under typical conditions until substantially complete whereupon the reaction is stopped and the Boc protected glutamine, 11, is recovered. Typical procedures for recovering the product include solvent stripping, chromatography, high performance liquid chromatography, precipitation, crystallization and the like. Alternatively, the product can be used in the next step without purification or isolation.

N-protected glutamine, 11, is then converted to the corresponding hydroxyamide by reaction with hydroxylamine in water (50%) using conventional methods well known in the art. The reaction proceeds under typical conditions until substantially complete whereupon the reaction is stopped and the hydroxyamide, 12, is recovered. Typical procedures for recovering the product include solvent stripping, chromatography, high performance liquid chromatography, precipitation, crystallization and the like. Alternatively, the product can be used in the next step without purification or isolation.

In the final step, the Boc protecting group is removed under acidic conditions well known in the art so as to provide for the glutamine amino acid mimetic, 4, which is recovered and purified in the manner described above.

In forming a dipeptide of formula II(a), the beta carboxyl group of glutamic or aspartic acid is blocked with a first protecting group and the amino group is blocked with a second protecting group wherein identical or orthogonal blocking groups can be used as shown below as both can be removed either simultaneously or sequentially:

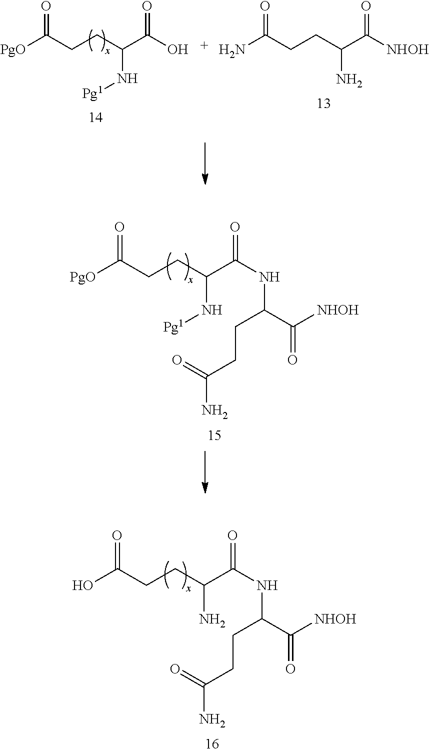

In the above scheme, well known and/or commercially available glutamic acid or aspartic acid blocked with conventional protecting groups at the beta carboxyl group and the amino group of compound 14 is covalently coupled to the glutamine mimetic 13 using well known amino acid coupling techniques such as DCC (dicyclohexylcarbodiimide) in pyridine or in an inert diluent with a suitable base. Suitable protecting groups are well known in the art and include Boc (t-butoxy-carbonyl), Cbz (carboxybenzyl), benzyl, and silyl protecting groups. For example, p-nitrobenzyloxy-C(O)CH₂CH₂CH(NHBoc)COOH is commercially available from SigmaAldrich, St. Louis, Mo., USA, as product number 5787469. The reaction is typically conducted until it is substantially complete whereupon the reaction is stopped and compound 15 is then recovered. Typical procedures for recovering the product include solvent stripping, chromatography, high performance liquid chromatography, precipitation, crystallization and the like. Alternatively, the product can be used in the next step without purification or isolation.

In the final step, the protecting groups are removed under conditions well known in the art so as to provide for the dipeptide incorporating the glutamine amino acid mimetic 16 that is recovered and purified in the manner described above.

Likewise, amidation at the beta carboxyl group of glutamic or aspartic acid is achieved merely by switching the carboxyl protecting group from the beta carboxyl group to the alpha carboxyl group and following the procedures set forth above. Suitably protected glutamic or aspartic acid groups are well known in the art and/or commercially available.

The addition of additional amino acids to the compounds of this invention can be achieved by conventional means whereby the amino acid(s) is/are inserted either between or after the glutamic acid residue. Such methods for growing oligopeptides are exceptionally well known in the art.

By following the procedures set for the above, the following compounds of this invention can be readily prepared (including salts, zwitterions, tautomers, anhydrides, and solvates thereof). All amino acids are in their L-conformation.

| Compound No. | Structure |
|---|---|
| A | 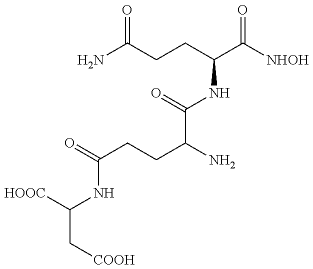 |
| B | 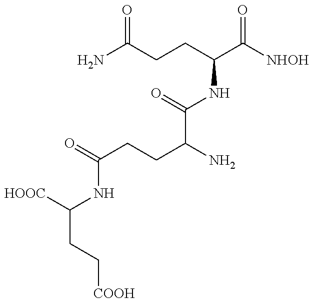 |
| C | 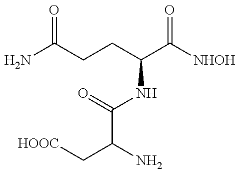 |
| D | 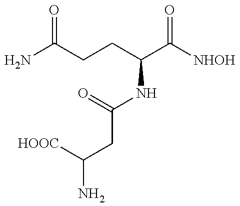 |
| E | 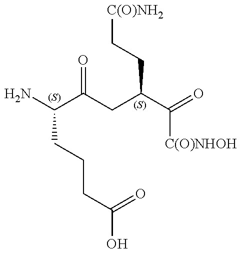 |
| F | 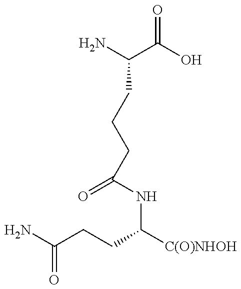 |
| G | 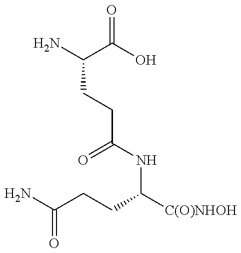 |
| H | 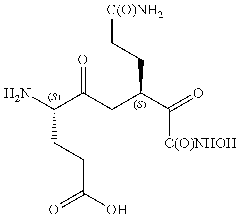 |

### Methods and Pharmaceutical Compositions

The compounds of this invention are useful in treating glutamine-dependent cancers. Such cancers are well known in the art and include, by way of example only, pancreatic cancer, leukemic cancers, triple negative breast cancer, and the like.

In particular, cellular amino acid transporters preferably absorb di- or tri-peptides that are then degraded intracellularly into individual amino acids. In addition, glutamine dependent cancers require substantially more glutamine than normal cells especially those cancers that overexpress glutamine transporters including but not limited to the SLA6A14 amino acid transporter. While glutamine is readily transported across the blood brain barrier, glutamic and aspartic acid are not as these are not neutral amino acids. In addition, the alteration of the carboxyl (COOH) group on glutamine to a hydroxyamide (NHOH) group acts to inhibit the glutaminase enzyme.

The above considerations have lead to glutaminase inhibitors that target glutamine dependent cancers but are inhibited in crossing the blood brain barrier due to the presence of glutamic or aspartic acid in the di- or tri-peptide structure. Accordingly, these compounds are useful in treating glutamine dependent cancers.

When so used, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors well-known to the skilled artisan. The drug can be administered at least once a day, preferably once or twice a day.

An effective amount of such inhibitors is readily ascertainable by simple IC₅₀ analyses. Various formulations and drug delivery systems are available in the art. See, e.g., Gennaro, A. R., ed. (1995) Remington's Pharmaceutical Sciences, 18^{th} ed. Mack Publishing Co.

As noted previously, an effective amount or a therapeutically effective amount or dose of an agent, e.g., a compound of this invention, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population), and the maximum tolerated dose (MTD), and the ED50 (the dose therapeutically effective in 50% of the population). The dose ratio of toxic to therapeutic effects is the therapeutic index, which can be expressed as the ratio LD50 or MTD/ED50. Agents that exhibit a high therapeutic index are preferred.

As also noted previously, the effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the cancer being treated, the sex, age, and weight of the subject being treated, the severity of the affliction, the manner of administration, and the judgment of the prescribing physician. In one embodiment, an effective amount of a compound of this invention is administered to the patient. Preferably, the effective amount of a compound of this invention ranges from about 0.1 mg/kg to about 75 mg/kg and preferably at from about 1 mg/kg to about 30 mg/kg given at standard intervals well-known in the art.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is intravenous using a dosage regimen that can be adjusted according to the degree of affliction. Other pharmaceutical compositions can take the form of tablets, pills, capsules, semisolids, powders, sustained release formulations, solutions, suspensions, elixirs, aerosols, or any other appropriate compositions.

The choice of formulation depends on various factors such as the mode of drug administration and bioavailability of the drug substance.

Pharmaceutical dosage forms of a compound of this invention may be manufactured by any of the methods well-known in the art, such as, for example, by conventional mixing, sieving, dissolving, melting, granulating, dragee-making, tableting, suspending, extruding, spray-drying, levigating, emulsifying, (nano-/micro-) encapsulating, entrapping, or lyophilization processes. As noted above, the compositions of this invention can include one or more physiologically acceptable inactive ingredients that facilitate processing of active molecules into preparations for pharmaceutical use.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of this invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semi-solid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention the in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The compositions of this invention may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compositions comprising a compound of this invention that can be formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt % of a compound of this invention based on the total formulation, with the balance being one or more suitable pharmaceutical excipients. Preferably, the compound is present at a level of about 1-80 wt %. Representative pharmaceutical formulations are described below.

### FORMULATION EXAMPLES

The following are representative pharmaceutical formulations containing a compound of this invention.

### Formulation Example 1—Tablet Formulation

The following ingredients are mixed intimately and pressed into single scored tablets.

| | Quantity per |
|---|---|
| Ingredient | tablet, mg |
| compound of this invention | 400 |
| Cornstarch | 50 |
| croscarmellose sodium | 25 |
| Lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Formulation Example 3—Suspension Formulation

The following ingredients are mixed to form a suspension for oral administration.

| Ingredient | Amount |
|---|---|
| compound of this invention | 1.0 g |
| fumaric acid | 0.5 g |
| sodium chloride | 2.0 g |
| methyl paraben | 0.15 g |
| propyl paraben | 0.05 g |
| granulated sugar | 25.0 g |
| sorbitol (70% solution) | 13.00 g |
| Veegum K (Vanderbilt Co.) | 1.0 g |
| Flavoring | 0.035 mL |
| Colorings | 0.5 mg |
| distilled water | q.s. to 100 mL |

### Formulation Example 4—Injectable Formulation

The following ingredients are mixed to form an injectable formulation.

| Ingredient | Amount |
|---|---|
| compound of this invention | 0.2 mg-20 mg |
| sodium acetate buffer solution, 0.4M | 2.0 mL |
| HC1 (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this invention with Witepsol® H-15 (triglycerides of saturated vegetable fatty acid; Riches-Nelson, Inc., New York), and has the following composition:

| Ingredient | Amount |
|---|---|
| Compound of this invention | 500 mg |
| Witepsol ® H-15 | balance |

The following synthetic and biological examples are offered to illustrate this invention and are not to be construed in any way as limiting the scope of this invention. Unless otherwise stated, all temperatures are in degrees Celsius.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims.

### Example 1—Synthesis of Compound 6

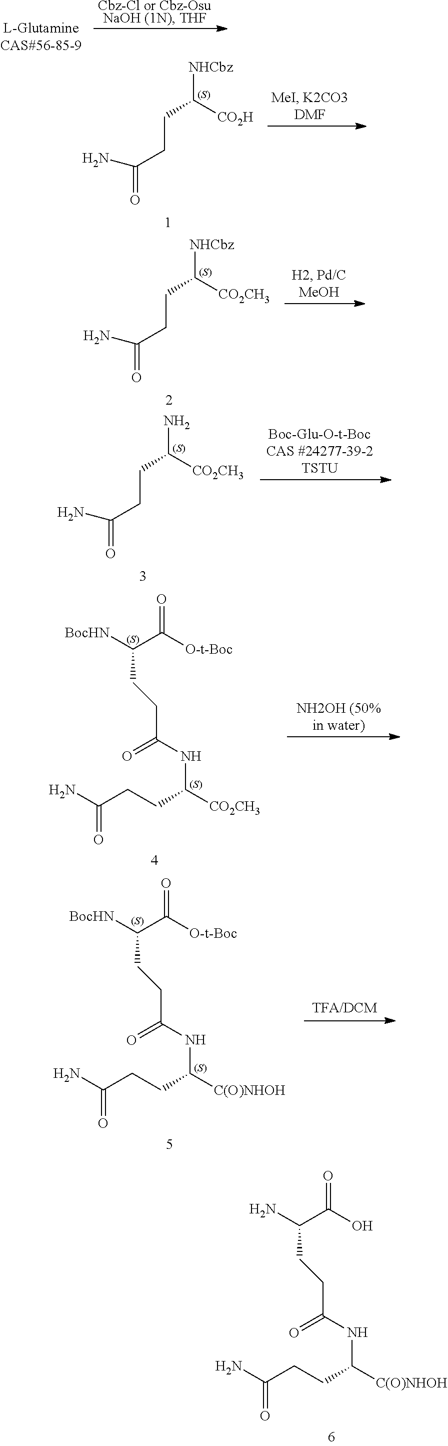

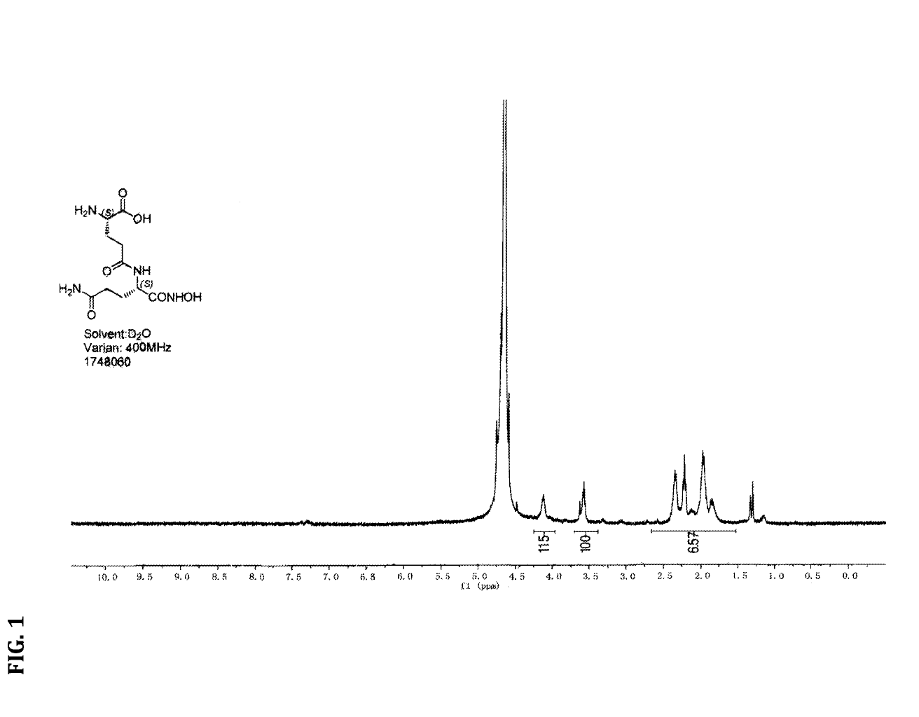

Compound 6 is prepared as described in detail above. Each of the above reactions, by itself, is well known in the art with the understanding that the combination is not. The NMR spectrum for Compound 6 is provided in FIG. 1.

### Example 2—Synthesis of Compound 9 and 10

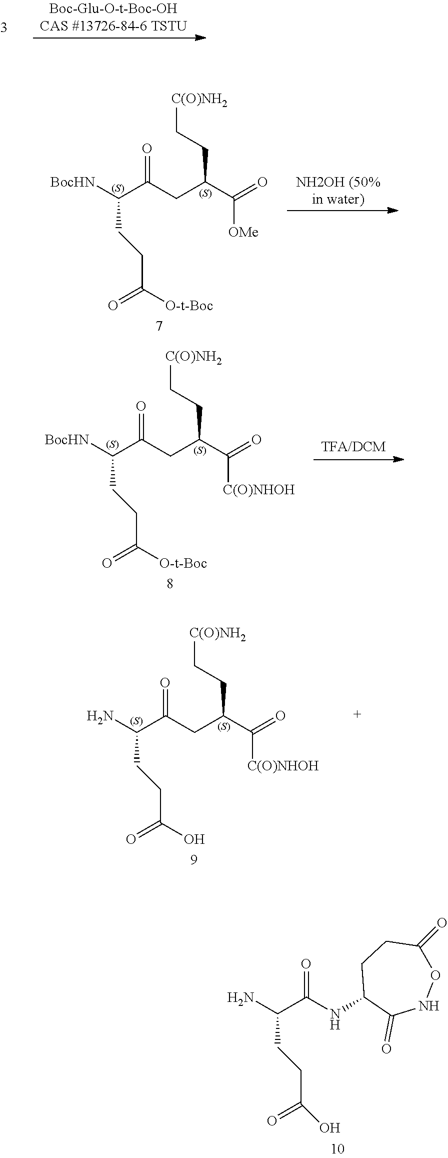

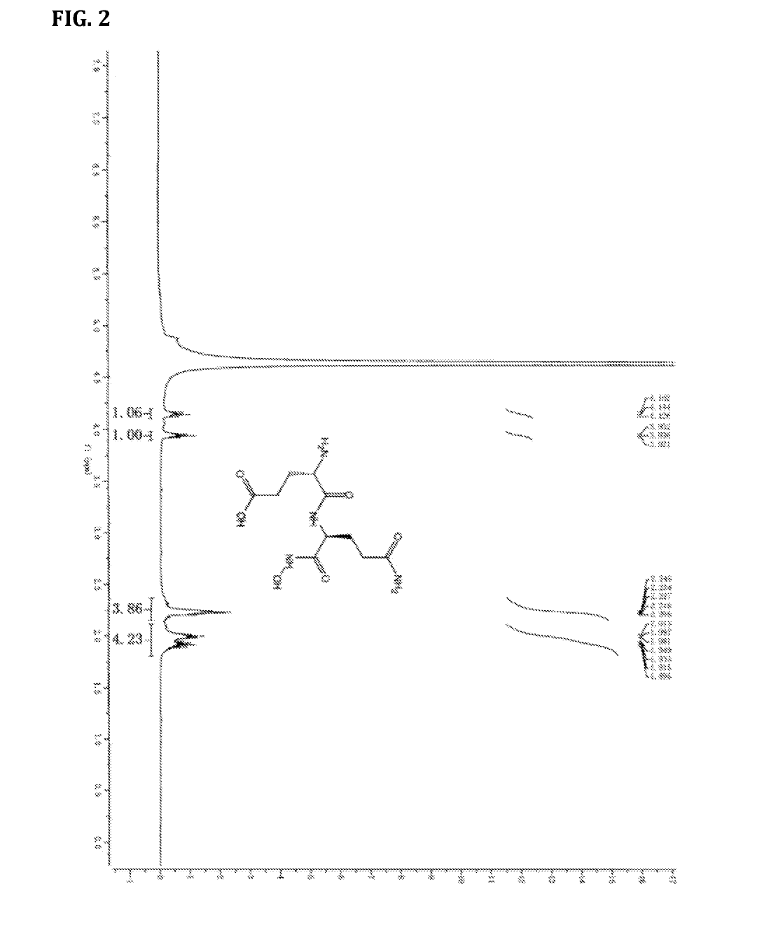

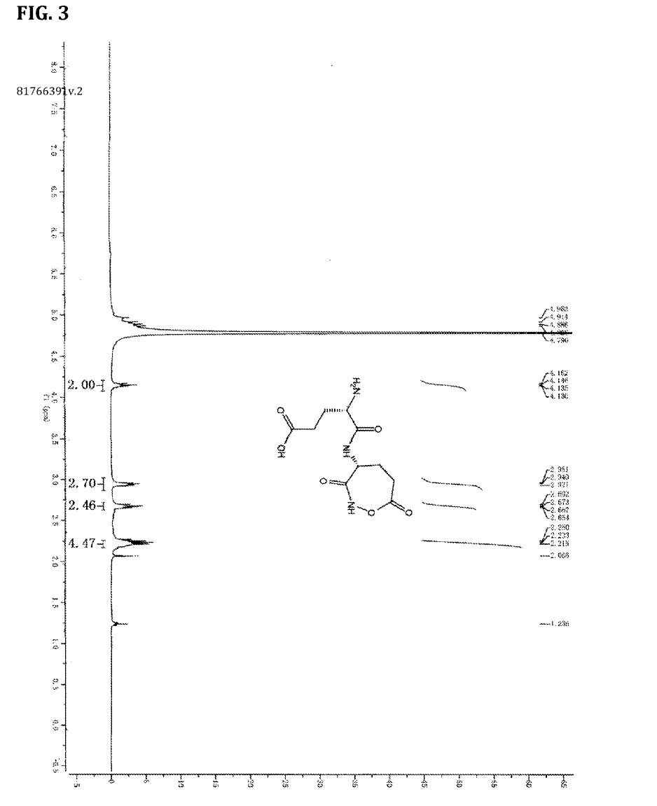

Compounds 9 and 10 are prepared as described in detail above. Each of the above reactions, by itself, is well known in the art with the understanding that the combination is not. The NMR spectrum for compound 9 is provided in FIG. 2 and that of 10 in FIG. 3.

### Biology

### Example 3—Compounds 6 and 9 Tested Against IM-9 Cells

Compounds 6 and 9 were tested for their ability to kill IM-9 cells (Multiple Myeloma). Approximately 25,000 cells were plated in RPMI medium (4 mM glutamine). Compounds 6 and 9 were dissolved in PBS and pH adjusted to neutrality (200 mM stock). Compound 9 was added at 1 mM, 2 mM and 4 mM concentrations into separate wells. Compound 6 was added at 0.25 mM and 1 mM concentrations into separate wells. The cells were incubated for 48 and 96 hours in the presence of compound 17, and 48 and 72 hours in the presence of compound 6. Cell number was determined with the CyQuant NF assay (measures DNA content) according to manufacturer's instruction (Invitrogen). The rates for cell kill against control are set forth in Tables 1 and 2 below. For the sake of clarity, compounds 6 and 9 are reproduced below:

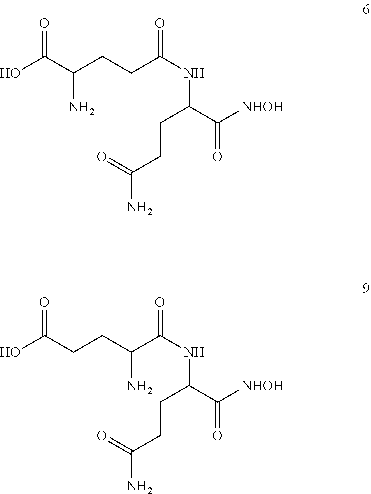

TABLE 1

| | Day 2 | Day 3 | Day 4 |
|---|---|---|---|
| Compound | Cells Killed v. | Cells killed v. | Cells killed v. |
| tested | control | control | control |
| 1 mM | ~17% | — | ~58% |
| compound 9 | | | |
| 2 mM | ~29% | — | ~66% |
| compound 9 | | | |
| 4 mM | ~61% | — | ~96% |
| compound 9 | | | |

TABLE 2

| | Day 2 | Day 3 | Day 4 |
|---|---|---|---|
| Compound | Cells killed v. | Cells killed v. | Cells killed v. |
| tested | control | control | control |
| 0.25 mM | ~77% | ~87% | — |
| compound 6 | | | |
| 1 mM | ~83% | ~91% | — |
| compound6 | | | |

The above data demonstrates that both compounds, when used at sufficient concentrations, killed significant numbers of the multiple myeloma cells. However, the data for compound 6 shows superiority both in the time required to kill these cells as well as at concentrations significantly less than those required for compound 9.

Indeed, at day 2, compound 6 at a concentration of 0.25 mM killed about 83% of the multiple myeloma cells as compared to only about 17% killed when compound 9 was used at a concentration of 1 mM. At day 4, compound 9 at a concentration of 4 mM provided for about a 96% death rate. On the other hand, compound 6 at 0.25 mM ( 1/16 of the concentration of compound 10) at day 3 provided for about a 91% death rate. To better illustrate these results, this data is set in a comparative format in Table 3 as follows:

TABLE 3

| | Day 2 | Day 3 | Day 4 |
|---|---|---|---|
| Compound | Cells killed v. | Cells killed v. | Cells killed v. |
| tested | control | control | control |
| 1 mM | ~17% | — | 58% |
| compound 9 | | | |
| 1 mM | ~83% | ~91% | — |
| compound 6 | | | |

### Example 4—Compounds 6 and 9 Tested Against Panc1 Cells

Compounds 6 and 9 were tested with Panc1 cells (Pancreatic cancer) plated in DMEM medium (2 mM glutamine). Approximately 30,000 live cells were plated. Compounds were dissolved in PBS and pH adjusted to neutrality (200 mM stock). Compound 9 was added at 1 mM, 2 mM and 4 mM concentrations into separate wells. Compound 6 was added at 0.25 mM and 1 mM concentrations into separate wells. The cells were incubated for 48 and 96 hours in the presence of compound 9, and 48 and 72 hours in the presence of compound 6. Cell number was determined with the CyQuant NF assay (measures DNA content) according to manufacturer's instruction (Invitrogen). Cell kill rates in each assay as compared to control are set forth in Tables 4 and 5 below.

TABLE 4

| | Day 2 | Day 3 | Day 4 |
|---|---|---|---|
| Compound | Cells Killed v. | Cells killed v. | Cells killed v. |
| tested | control | control | control |
| 1 mM | ~0% | — | ~6% |
| compound 9 | | | |
| 2 mM | ~7% | — | ~9% |
| compound 9 | | | |
| 4 mM | ~11% | — | ~55% |
| compound 9 | | | |

TABLE 5

| | Day 2 | Day 3 | Day 4 |
|---|---|---|---|
| Compound | Cells killed v. | Cells killed v. | Cells killed v. |
| tested | control | control | control |
| 0.25 mM | ~32% | ~50% | — |
| compd 6 | | | |
| 1 mM | ~66% | ~75% | — |
| compound 6 | | | |

The above data demonstrates that both compounds, when used at a sufficient concentrations, killed significant numbers of the pancreatic cancer cells. However, the data for compound 6 shows superiority both in the time required to kill these cells as well as at concentrations significantly less than those required for compound 9. Indeed, at day 4, compound 9 at a concentration of 1 mM killed about 6% of these pancreatic cancer cells. This compares to 75% of dead pancreatic cancer cells at day 3 for compound 6 when used at the same concentration. To better illustrate these results, this data is set in a comparative format in Table 6 as follows:

TABLE 6

| | Day 2 | Day 3 | Day 4 |
|---|---|---|---|
| Compound | Cells killed v. | Cells killed v. | Cells killed v. |
| tested | control | control | control |
| 1 mM | ~0% | — | 6% |
| compound 9 | | | |
| 1 mM | ~66% | ~75% | — |
| compound 6 | | | |

1. A compound of the formula: 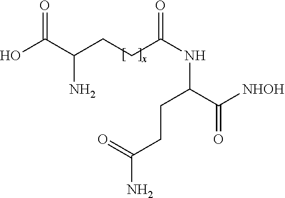
wherein x is zero, one, two or three and including pharmaceutically acceptable salts, tautomers, and zwitterions.

2. A pharmaceutical composition comprising an effective amount of a compound of claim 1 and a pharmaceutically acceptable excipient.

3. A method for killing multiple myeloma cells which method comprises contacting said cells with an effective amount of a compound of claim 1.

4. A method for killing multiple myeloma cells which method comprises contacting said cells with an effective amount of a pharmaceutical composition of claim 2.

5. A compound of the formula: 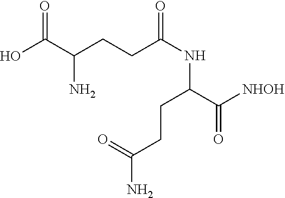
including pharmaceutically acceptable salts, zwitterions, and tautomers thereof.

6. A pharmaceutical composition comprising an effective amount of a compound of claim 5 and a pharmaceutically acceptable excipient.

7. A method for killing multiple myeloma cells which method comprises contacting said cells with an effective amount of a compound of claim 5.

8. A method for killing multiple myeloma cells which method comprises contacting said cells with an effective amount of a pharmaceutical composition of claim 6.

9. A method for killing pancreatic cancer cells which method comprises contacting said cells with an effective amount of a compound of claim 1.

10. A method for killing pancreatic cancer cells which method comprises contacting said cells with an effective amount of a pharmaceutical composition of claim 2.

11. A method for killing pancreatic cancer cells which method comprises contacting said cells with an effective amount of a compound of claim 5.

12. A method for killing pancreatic cancer cells which method comprises contacting said cells with an effective amount of a pharmaceutical composition of claim 6.
